(19) United States
(12) Patent Application Publication
(10) Pub. No.: US 2024/0277700 A1
(43) Pub. Date: Aug. 22, 2024

(54) COMPOSITIONS AND METHODS FOR TREATING AND PREVENTING INTERSTITIAL CYSTITIS

(71) Applicants: Reed B. HOGAN, II, Jackson, MS (US); Thomas P. DOOLEY, Jackson, MS (US); Douglas R. PAUL, Jackson, MS (US); Hista RX LLC, Jackson, MS (US)

(72) Inventors: Reed B. Hogan, II, Jackson, MS (US); Thomas P. Dooley, Pinson, AL (US); Douglas R. Paul, Oxford, MS (US)

(21) Appl. No.: 18/693,500

(22) PCT Filed: Sep. 21, 2022
(86) PCT No.: PCT/US2022/044283

§ 371 Date: Mar. 19, 2024

Related U.S. Application Data
(60) Provisional application No. 63/247,198, filed on Sep. 22, 2021.

(51) Int. Cl. A61K 31/495; A61K 31/426; A61P 13/10

(52) U.S. Cl. CPC A61K 31/495; A61K 31/426; A61P 13/10

(57) ABSTRACT

Pharmaceutical compositions and methods of treating or preventing interstitial cystitis of the bladder or the symptoms thereof in a patient that includes administering an H1 receptor antagonist and an H2 receptor antagonist to the patient.

### BACKGROUND

Histamine plays fundamental roles in modulating inflammation through increased capillary blood flow and vascular permeability, as well as cytokine release (Branco, et al. 2018; Kmiecik, et al. 2012). Mast cells are the hosts for histamine, which can be released into the extracellular environment via mast cell degranulation (Akin, et al. 2010; Hamilton, et al. 2011; Valent 2013). Histamine-1 (H1) receptor antagonists (e.g., cetirizine) are administered for allergies. Histamine-2 (H2) receptor antagonists (e.g., famotidine) are administered to control acid in the stomach and heart burn.

Prescription branded, generic, and over-the-counter (OTC) drugs of both classes of antihistamines are commercially available essentially worldwide. The OTC H1 and H2 antihistamines (e.g., cetirizine and famotidine, respectively) are deemed as exceptionally safe by the US Food and Drug Administration (FDA).

Treatment of diseases using specific combinations that include an H1 receptor antagonist and an H2 receptor antagonist have been attempted or proposed: urticaria (Lin, et al. 2000; Monroe, et al. 1981; Phanuphak, et al. 1978; Wan 2009; Runge, et al. 1992), diarrhea (Hogan I I 2014; Hassoun, et al. 2019; Mohammadi, et al. 2018); morphine anesthesia side effects (Philbin, et al. 1981); and exercise physiology (Pellinger, et al. 2010). In 2020 Hogan and coworkers reported that a cohort of 110 severe and critical patients hospitalized with pulmonary symptoms of SARS-COV2/COVID-19 can be effectively and safely treated with cetirizine plus famotidine (Hogan I I, et al. 2020). It has been proposed that the benefit of cetirizine 10 mg and famotidine 20 mg twice daily is due to reducing the pulmonary inflammatory “cytokine storm” downstream of histamine's action, which is common in patients with severe to critical symptoms (Mehta, et al. 2020).

A porcine animal model study has shown successful treatment of Pseudomonas-induced ARDS with diphenhydramine (H1) and cimetidine (H2) (Sielaff, et al. 1987). In this model the combination of diphenhydramine and cimetidine was essential and was augmented somewhat by ibuprofen (NSAID). In a guinea pig model, treatment with clemastine (H1) and cimetidine (H2) protected against allergen-induced bronchial obstruction (Dorsch, et al. 1982).

Interstitial cystitis (IC) is an inflammatory disorder of the bladder. The primary clinical symptom of IC is chronic pain of the bladder (and/or pelvic region), and the disorder is sometimes termed as “bladder pain syndrome” or “painful bladder syndrome”. Associated with the pain are increased frequency of urination when awake and/or while sleeping or attempting to sleep (i.e., nocturia), and/or urinary urgency. These symptoms and others (e.g., urinary incontinence, mental anxiety) associated with IC can result in substantial disruptions of normal activities (e.g., the abilities to work, exercise, sleep, concentrate, to enjoy sexual intercourse, etc.).

Diagnosis of IC can be problematic, as there are no specific tests to affirm a diagnosis. Urologists, gynecologists, or other physicians may use cystoscopy to internally examine the bladder for erythema (redness) and swelling, and to exclude other possible reasons for the pain (e.g., urinary tract infection or cancer). Ultrasound imaging might also be used in confirming a diagnosis of IC.

Histamine has been strongly implicated in the pathogenesis of human IC. Methylhistamine and histamine are biomarkers (el-Mansoury, et al. 1994; Lamale, et al. 2006; Yun, et al. 1992), and histamine receptors gene expression has been evaluated (Shan, et al. 2019). Mast cell counts and physiology have been studied in this inflammatory disease (Enerback, et al. 1989; Kastrup, et al. 1983; Lundeberg, et al. 1993; Lynes, et al. 1987). Monotherapies affecting histamine levels or receptor binding have been used to treat IC. Pentosan polysulfate is an active pharmaceutical ingredient (API) used in the treatment of IC, and it affects histamine release (Chiang, et al. 2000). Hydroxyzine (H1 receptor antagonist) has been used with some limited benefit (Sant, et al. 2003; Theoharides 2007). And, high dose cimetidine (H2 receptor antagonist) monotherapy at high doses of 300 mg bid (600 mg daily) for one month was effective at symptomatic relief in a case series report of 9 patients (Seshadri, et al. 1994). Multimodal therapy has been proposed to be more effective than monotherapies in the treatment of IC (Evans 2002).

In spite of frontline SOC medications and procedures, many patients' symptoms remain refractory to treatment for months and years. Thus, there is a need for a safe and effective treatment and/or prophylaxis for IC or the symptoms thereof. Dual histamine receptor blockade provides a solution to this problem, and utilizing historically exceptionally safe pharmaceutical active ingredients.

### SUMMARY

In a first aspect, the present invention is a method of treating or preventing interstitial cystitis in a patient, including administering an H1 receptor antagonist and an H2 receptor antagonist to the patient.

In a second aspect, the present invention is a method of treating or preventing bladder pain, frequent urination, or urinary urgency in a patient affected by interstitial cystitis, including administering an H1 receptor antagonist and an H2 receptor antagonist to the patient.

In a third aspect, the present invention is a method of treating or preventing inflammation of the bladder in a patient, including administering an H1 receptor antagonist and an H2 receptor antagonist to the patient.

In a fourth aspect, the present invention is a pharmaceutical composition for use in treating or preventing interstitial cystitis or symptoms thereof in a patient. The pharmaceutical composition includes an H1 receptor antagonist and an H2 receptor antagonist.

In a fifth embodiment, the present invention is the use of an H1 receptor antagonist and an H2 receptor antagonist for the preparation of a medicament for treating or preventing interstitial cystitis in a patient.

### Definitions

The term “unit dosage form,” means a single pre-measured dose, and includes tablets, pills, capsules, packets, suspensions, transdermal patches, rectal suppositories, and sterile pre-measured liquid formulations ready for injection.
H1 means histamine type-1.
H1R means histamine type-1 receptor.
H2 means histamine type-2.
H2R means histamine type-2 receptor.
IC means interstitial cystitis.
OTC means over-the-counter.
IV means intravenous administration.
PO means oral administration.
The abbreviation, “bid” means twice daily.
GMP means Good Manufacturing Practice.
SOC means standard(s)-of-care.
API means active pharmaceutical ingredient.
ODT means orally-disintegrating tablet.

The letter “q” relates to frequency of administration. For example, qD means once a day. Q8 hr. means a dose of medicine is administered every 8 hours.

### DETAILED DESCRIPTION

The present invention includes methods of using a histamine H1 receptor antagonist and a histamine H2 receptor antagonist for the treatment and prophylaxis of interstitial cystitis. IC is a chronic painful bladder condition that may manifest along with frequent urination, urinary urgency, and/or pelvic pain. It is more common in women (i.e., ca. 90 percent) than in men. Patients experiencing bladder pain and/or the need for frequent and/or urgent urination may be treated by administering an H1 receptor antagonist and an H2 receptor antagonist. Administering an H1 receptor antagonist and an H2 receptor antagonist has been shown to be effective at treating IC, reducing the level of pain, and reducing the frequency of urination.

H1 receptor antagonists and H2 receptor antagonists as monotherapieshave been shown to be exceptionally safe, and examples of each class have warranted governmental registrations with over-the-counter (OTC) status. Based upon historic clinical evidence in the treatment of other human medical conditions, administering an H1 receptor antagonist and an H2 receptor antagonist is anticipated to be safe for patients, will have few potential side effects, and may be a preferred treatment or prophylaxis over clinical standard(s)-of-care (SOC) treatments (e.g., NSAIDs, invasive procedures).

An effective treatment by H1 and H2 receptor antagonist combination therapy: (a) could reduce the severity of the primary symptom, i.e., pain generated within the bladder; (b) could reduce incontinence, urination urgency, and/or frequency of urination; (c) might beneficially affect the structure and health of bladder tissue in ways that are evident by cystoscopy and/or ultrasound imaging (e.g., reduction in inflammation); and/or (d) could improve quality of life parameters, such as the abilities to work, exercise, sleep, and concentrate, among others.

An effective treatment of IC by H1 and H2 receptor antagonist combination therapy could be administered for short duration (e.g., 1-7 days) acute episodes. This combination therapy could also be administered daily for long duration (weeks, months, or even years) for prophylaxis or maintenance.

The historic safety in humans around the globe for antihistamines, such as cetirizine and famotidine, provide a distinct advantage to the IC combination therapy of the present invention. OTC approval and designation by a regulatory agency, such as the US FDA, is merited for only the safest of medications in view of historic pharmaceutical surveillance. OTC use in the general population provides a heightened level of safety, and a reduced safety concern regarding drug-drug interactions. Millions of patients in the US alone routinely administer OTC H1 or H2 receptor antagonist medications effectively and safely.

A physician or physicians may use their professional skills and experiences in diagnosis, treatment, and prophylaxis of interstitial cystitis using H1 and H2 receptor antagonist combination for the treatment and/or prophylaxis of a patient or patients afflicted by IC. The historic evidence of safe and effective combination therapy with dual antihistamine APIs in other human diseases, as well as the exceptionally safe and relatively low doses of each API are important considerations for writing prescriptions for the medication.

A patient or patients can be treated with the dual histamine receptor antagonist therapy and perceive benefits with regard to their IC symptoms, such as reduced bladder pain, urinary urgency, and/or frequency of urination. A patient or patients does not perceive adverse side effects that would warrant discontinuation of the therapy. Some patients might perceive light sedation as a minor side effect of H1 receptor antagonists, although this is well tolerated.

Given the historic safety and especially the US FDA approved OTC status of H1 and H2 receptor antagonist exemplars, cetirizine and famotidine, respectively, combinations thereof at doses similar to the approved OTC doses as monotherapies could be preferred embodiments of the present invention, including for long duration therapies. Furthermore, a preferred embodiment could be combinations of H1 and H2 receptor antagonists (e.g., cetirizine and famotidine, respectively) that do not exceed the dosages of the FDA (or other regulatory agency) approved OTC products as monotherapies.

The present invention makes use of administering an H1 receptor antagonist and an H2 receptor antagonist in combination to a patient, to provide prevention of or a significant reduction of symptoms of interstitial cystitis. Preferred H1 receptors include cetirizine, levocetirizine, and diphenhydramine. Preferred H2 receptors include famotidine and ranitidine. Administration orally (p.o.) is preferred.

The safety of antihistamine agents is also advantageous. The historic safety of selected H1R and H2R antagonists (e.g., cetirizine, diphenhydramine, famotidine) enabled formulations with these active pharmaceutical ingredients (APIs) to be regulated as over-the-counter (OTC) medications. For instance, cetirizine is a US OTC at 10 mg, diphenhydramine is a US OTC at 25 mg, and famotidine is a US OTC at 10 or 20 mg. The OTC safety designations, coupled to the efficacy and safety profile of dual-histamine blockade in a variety of contexts in animal models and humans to date, makes this an appealing consideration for all patients.

Furthermore, the safety profile of the preferred histamine receptor antagonists in this combination drug approach provides distinct advantages over the clinical standard(s)-of-care treatments (e.g., NSAIDs). A safe and effective approach is facilitated by the dual-histamine blockade for IC symptoms, relative to current standard(s)-of-care.

The average weight of an adult in North America is approximately 80 kg, with the male average above this level and the female average below this level. The average weight of a 12-year-old child is approximately 40 kg, with males and females having a similar average weight. Therefore, a dose of 10 mg of a pharmaceutical agent administered to an average adult is 0.125 mg/kg and an equivalence dose of 5 mg administered to an average 12-year-old child is also 0.125 mg/kg, presuming no allometric scaling factor for weight differential. Thus, the equivalent dose administered to a 12-year-old child would be expected to be one half of that of the adult dose.

The present invention also includes unit dosage forms, multi-dosage forms, and kits, including an H1 receptor antagonist and an H2 receptor antagonist. Preferably, the H1 receptor antagonist includes cetirizine and the H2 receptor antagonist includes famotidine. The combination may be an oral dosage form for ingestion, or a gastric dosage form for delivery via a nasogastric tube, a trans-urethral infusion, a mucosal dosage form, or an injectable dosage form (e.g., IV).

H1 receptor antagonists block H1 histamine receptors; first-generation H1 receptor antagonists block histamine receptors in the central and peripheral nervous systems, as well as cholinergic (muscarinic) receptors, while second-generation H1 receptor antagonists are selective for H1 histamine receptors in the peripheral nervous system. First-generation H1 receptor antagonists include brompheniramine, chlorpheniramine, dexbrompheniramine, dexchlorpheniramine, pheniramine, triprolidine, carbinoxamine, clemastine, diphenhydramine, pyrilamine, promethazine, hydroxyzine, azatadine, cyproheptadine, and phenindamine. Second-generation H1 receptor antagonists include ketotifen, rupatadine, mizolastine, acrivastine, ebastine, bilastine, bepotastine, terfenadine, quifenadine, azelastined, cetirizine, levocetirizine, desloratadine, fexofenadine and loratadine. Preferably, the H1 receptor antagonist is a second-generation H1 receptor antagonist, more preferably the H1 receptor antagonist is cetirizine or levocetirizine, with cetirizine being particularly preferred. Mixtures and combination of H1 receptor antagonists may also be used.

Prescription generic and over-the-counter (OTC) products containing at least one H1R antagonist are currently FDA approved and commercially-available. Prescription generic and/or OTC products containing at least one H1R antagonist may be preferred for specific medical conditions of the present invention. For instance, generic and/or OTC products may be preferred for outpatient treatments.

The H1 receptor antagonists may be used in an amount of from 0.1 to 10 times the amount typically used for the treatment of allergies, for example in an amount of 0.1 to 600 mg per dose, 0.5 to 500 mg per dose, 1.0 to 50 or 60 mg per dose, including 1.25, 1.5, 1.75, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40 and 45 mg per dose. Preferably, the H1 receptor antagonist is administered 1, 2, 3 or 4 times per day. The H1 receptor antagonist may be administered as an injectable formulation, for example intravenously, intraparenterally or intramuscularly; transdermally, via a transdermal patch; or, preferably, orally, as a powder, table or capsule, an oral solution or suspension, or sublingual or buccal tablets; or orally-disintegrating tablet (ODT); or preferably a solution or suspension via trans-urethral canula or nasogastric tube. Alternative forms of administration include rectal suppositories, inhaled, subcutaneous, nasal spray, transmucosal, and intradermal formulations.

H2 receptor antagonists block H2 histamine receptors. H2 receptor antagonists include cimetidine, ranitidine, famotidine, and nizatidine, with famotidine being preferred. Mixtures and combinations of H2 receptor antagonists may also be used.

Prescription generic and over-the-counter (OTC) products containing at least one H2R antagonist are currently FDA approved and commercially-available. Prescription generic and/or OTC products containing at least one H2R antagonist may be preferred for specific medical conditions of the present invention. For instance, generic and/or OTC products may be preferred for outpatient treatments.

The H2 receptor antagonists may be used in an amount of from 0.1 to 10 times the amount typically used for treatment dyspepsia, for example 1.0 to 8000 mg per dose, 2.0 to 1000 mg per dose, 5.0 to 800 mg per dose, including 6.0, 7.0, 8.0, 9.0, 10, 15, 20, 21, 22, 22.5, 23, 24, 25, 26, 27, 28, 29, 30, 35, 40, 45, 50, 55, 60, 65, 70, 75, 80, 85, 90, 95, 100, 120, 140, 150, 175, 200, 250, 300, 350, 400, 450, 500, 600, and 700 mg per dose. Preferably, the H2 receptor antagonist is administered 1, 2, 3 or 4 times per day. The H2 receptor antagonist may be administered as an injectable formulation, for example intravenously, intraparenterally or intramuscularly;

transdermally, via a transdermal patch; or, preferably, orally, as a powder, table or capsule, an oral solution or suspension, or sublingual or buccal tablets; or ODT; or preferably a solution or suspension via trans-urethral canula or via nasogastric tube. Alternative forms of administration include rectal suppositories, inhaled, subcutaneous, nasal spray, transmucosal, and intradermal formulations.

Patients may respond to treatment within several days to several weeks. However, treatment should be carried out for an amount of time to resolve the symptoms (e.g., pain), for example 3 to 30 days, or 6 to 60 days. Treatment may be initiated at the first signs of pain or thereafter. Patients may administer the pharmaceutical composition for the prophylaxis of IC symptoms, for instance following successful treatment (e.g., reduced symptom severity) by the pharmaceutical composition or another SOC treatment (e.g., medication or procedure).

Preferably, the H1 and H2 receptor antagonists are administered together or simultaneously, and as a unit dosage form containing both receptor antagonists. The H1 receptor antagonist and the H2 receptor antagonist may also be administered sequentially, for example, at intervals ranging from 1 to 12 hours between each administration. Examples of unit dosage forms include oral compositions, such as tablets (for example, sublingual or buccal tablets), two-sided tablets, uncoated or coated tablets, orally disintegrating tablets (ODTs), capsules (for example, hard gelatin and soft gelatin capsules), transmucosal and sublingual patches and films, pre-measured powder packets and sachets, flavored and/or sweetened aqueous solutions or suspensions. Preferably, the unit dosage form is present as a once-per-day dosage, although other dosage schedules are envisioned (e.g., twice-per-day).

Various pharmaceutical formulations containing H1 and H2 receptor antagonist API combinations are envisioned. Examples include compressed tablets, uncoated and coated tablets, two-sided compressed tablets, gelatin capsules filled with powder, aqueous liquids and suspensions for oral and other routes of administration, alcoholic elixirs, liquids or suspensions for trans-urethral bladder infusion, orally disintegrating tablets (ODTs) with and without taste masking and flavoring agents, among others.

Other dosage forms include aqueous liquids and suspensions for oral and other routes of administration, and alcoholic elixirs, either with and without flavoring or taste-masking.

Other unit dosage forms may also be provided, containing both H1 and H2 receptor antagonists. For example, an injectable formulation containing a sterile solution or suspension, including formulation for administration intravenously, intraparenterally or intramuscularly, may be provided. An injectable formulation, such as a sterile premeasured single dose ready-to-inject form is also a unit dosage form. A unit dosage of a solution or suspension may be delivered by nasogastric tube into the stomach. A unit dosage form for administration transdermally, via a transdermal patch, may be provided. Other unit dosage forms include rectal suppositories, inhaled, epidural, subcutaneous, nasal spray, and intradermal formulations.

Other dosage forms may include aqueous liquids and suspensions for trans-urethral bladder infusion via a urinary canula.

Pharmaceutically acceptable carriers, excipients and adjuvants (e.g., buffers, surfactants, lipophilic agents, sugars, alcohols, solubilizers, bonding agents, dispersion agents, and/or pharmaceutically acceptable salts thereof) may also be included in any of the pharmaceutical compositions or unit dosage forms, both oral and non-oral.

Multi-dosage forms, such as kits, containing 2 to 30, 3 to 25, or 5 to 14 unit dosage forms, for example 6, 7, 8, 9, 10, 11, 12, 13, 15, 20, 40, 50 or 60 unit dosage forms, may be provided. Preferably, the multi-dosage forms contain sufficient unit dosage forms for administration over a period of 2 to 30, 3 to 25, or 7 to 14 days, for example 4, 5, 6, 7, 8, 9, 10, 11, 12, 13, 20 or 30 days. Kits may also be provided, which include oral rehydration solutions, or powders which may be hydrated to form oral rehydration solutions, or kits containing sodium and glucose or a glucose-containing saccharide, as well as other pharmaceutically acceptable excipients, flavorings and/or sweeteners, buffers, together with unit dosage forms.

Histamine H1 receptor antagonists include but are not limited to acrivastine, alimemazine, antazoline, astemizole, azatadine, azelastine, bamipine, bromazine, brompheniramine, buclizine, carbinoxamine, cetirizine, chlorcyclizine, chloropyramine, chlorphenamine, chlorpheniramine, chlorphenoxamine, cinnarizine, clemastine, cyclizine, cyproheptadine, deptropine, desloratadine, dexbrompheniramine, dexchlorpheniramine, dimenhydrinate, dimetindene, diphenhydramine, diphenylpyraline, dithiaden, doxepin, doxylamine, ebastine, emedastine, epinastine, fexofenadine, histapyrrodine, hydroxyethylpromethazine, hydroxyzine, isothipendyl, ketotifen, levocabastine, levocetirizine, loratadine, mebhydrolin, meclizine, meclozine, medizine, mepyramine, mequitazine, methapyrilene, methdilazine, mizolastine, mizolastine, olopatadine, oxatomide, oxomemazine, phenindamine, pheniramine, pimethixene, promethazine, pyrilamine, pyrrobutamine, rupatadine, talastine, terfenadine, thenalidine, tripelennamine, and triprolidine. Combinations and mixtures of H1 receptor antagonists may be used.

Histamine H2 receptor antagonists include but are not limited to cimetidine, famotidine, nizatidine, ranitidine, roxatidine, ebrotidine, lafutidine, niperotidine, potentidine, and zolantidine. Combinations and mixtures of H2 receptor antagonists may be used.

### EXAMPLES

### Example 1: Compounded Pharmaceutical Formulations

A nonsterile compounded pharmaceutical formulation was prepared according to US FDA 503A regulations. Number 1 gelatin capsules were prepared by extemporaneous compounding containing Cetirizine HCl 8 mg and Famotidine 22 mg as APIs, plus inert pharmaceutical excipients (e.g., lactose, etc.). The selected doses are close to, or similar to, but not identical to, the US FDA approved doses for each API as OTC monotherapies (i.e., Cetirizine HCl at 10 mg and Famotidine at 10 or 20 mg).

Additional API combinations, dosage forms, and selected doses are possible for 503A and 503B compounding.

### Example 2: A Patient with Chronic Interstitial Cystitis and Treated Effectively with Dual-Histamine Receptor Blockade

A 25-year-old female with a medical history of at least 8 years and diagnoses of interstitial cystitis, chronic IBS-diarrhea (IBS-D), postural orthostatic tachycardia syndrome (POTS), and possibly Crohn's disease, had previously taken multiple nonsteroidal anti-inflammatory drugs (NSAIDs), Esomeprazole (proton pump inhibitor), and Sucralfate (antacid), and without resolution of her urologic and/or gastrointestinal conditions. The patient experienced painful frequent urination 15-20 times per day with incontinence. She described the cystitis and diarrhea symptoms as 9/10 in severity with significant lifestyle disruption. She was unable to sleep through the night and experienced total disruption of her job. There were no noteworthy irregularities in clinical chemistry or hematology.

She was re-assessed by another physician for the uncontrolled IBS-diarrhea and was prescribed a compounded pharmaceutical formulation (US FDA 503A) of Cetirizine 8 mg plus Famotidine 22 mg in an orally ingested gelatin capsule that was administered once daily. Note that the selected doses of the APIs are under the maximum daily doses for each active ingredient as US FDA-approved OTC medications, namely Cetirizine at 10 mg maximum daily and Famotidine at 40 mg maximum daily.

Thereafter the patient experienced substantial reduction in her cystitis and gastrointestinal symptoms, which prior to that date had not been achieved by any NSAIDs or other medications. As further evidence of the beneficial effects, the patient requested and received multiple refills of the compounded H1 and H2 receptor antagonist combination medication. Her cystitis symptoms were markedly improved, however it recurred whenever she stopped the prescription compounded H1 and H2 receptor antagonist combination therapy. Using this treatment her cystitis no longer interfered with her job or lifestyle.

Approximately three years later by verbal interview she described her improvement as “life changing” and reported “at most a 2/10” symptom severity for both IC and IBS-D. The patient's symptoms recurred if she discontinued treatment with the H1 and H2 receptor antagonist combination medication for 4-5 days. She continued to obtain refills of the compounded medication. She stated, “I tell all my (location) friends about my miracle medicine.” This has been the only effective treatment and prophylaxis for her persistent IC symptoms.

### Example 3: A Patient with Chronic Painful Bladder Syndrome and Treated Effectively with Dual-Histamine Receptor Blockade

A 47-year-old female with a diagnosis of at least 5 years duration of painful bladder syndrome manifested pain in the bladder, vagina, occasionally bilateral flanks, vestibulodynia, and cramping. Her urinary pathology was negative; thus, the pain was not due to a urinary tract infection. To attempt to alleviate the symptoms she had previously taken Phenazopyridine, the H1 antihistamine Hydroxyzine, and/or Acetaminophen prn. The patient experienced severe urinary urgency resulting in frequent urination once per hour, once nightly nocturia, and without incontinence.

She was prescribed a compounded pharmaceutical formulation (US FDA 503A) of Cetirizine 8 mg plus Famotidine 22 mg in an orally ingested gelatin capsule that was administered once daily, in addition to Calcium Glycerophosphate for regulation of dietary acid prn, while continuing to use Hydroxyzine. Note that Hydroxyzine, with both H1 antihistamine and antimuscarinic activities, is often prescribed for IC and/or anxiety.

At follow up 6 weeks thereafter the patient reported that she had experienced substantial reduction in her bladder pain symptoms while taking the Cetirizine-Famotidine combination. As further evidence of the beneficial effects of the H1+H2 receptor antagonist combination, the patient reported a reduction in urinary frequency to every 2.5 hours, no nocturia, and only mild urgency. This level of improvement had not been achieved by any of the prior medications. She continued to administer the compounded Cetirizine-Famotidine drug combination thereafter, as it provided symptomatic relief, whereas prior treatments, such as the H1 antihistamine Hydroxyzine, had been insufficient.

At follow up 6 months later she stated that she is doing very well and feels “normal”. Urinary frequency is every 2 hours with no nocturia, with normal urinary urgency. She was advised to continue the Cetirizine-Famotidine combination and Hydroxyzine.

### Example 4: A Patient with Severe Bladder Pain and Overactive Bladder and Treated Effectively with Dual-Histamine Receptor Blockade

A 57-year-old female manifested symptoms of multi-year duration of bladder pain and overactive bladder, among other physiologic and mental health disorders. At an office visit one year prior the patient expressed that she had experienced severe and painful urinary urgency resulting in frequent urination once per 0.5 hour (30 minutes), nocturia 6 times per night, and without incontinence.

Multiple urologic and pain medications (e.g., Uribel, Gabapentin, Prelief, Oxybutynin, Phenazopyridine, Oxycodone, Pentosan Polysulfate, Dicyclomine, and the H1 receptor antagonist Loratidine) and procedures (e.g., bladder instillations, cystoscopy with hydrodistention, and Botox injections) were attempted to address her bladder disorder. However, polypharmacy and the multiple procedures failed to ameliorate the severity of this patient's chronic disease state.

Therefore, H1+H2 histamine receptor blockade was attempted for this recalcitrant condition. She was prescribed a compounded pharmaceutical formulation (US FDA 503A) of Cetirizine 8 mg plus Famotidine 22 mg in an orally ingested gelatin capsule that was administered once daily, in addition to Hydroxyzine. Note that Hydroxyzine, with both H1 antihistamine and antimuscarinic activities, is often prescribed for IC and/or anxiety.

At follow up 3 months thereafter the patient reported that she had experienced reduction in her bladder pain while taking the Cetirizine-Famotidine combination. As further evidence of the beneficial effects of the H1+H2 receptor antagonist combination, the patient reported a reduction in urinary frequency to every 1.5 hours, nocturia 4-5 times nightly, and moderate urgency. She continued to administer the compounded dual drug combination thereafter, and Botox injections continued.

At follow up 9 months after starting Cetirizine-Famotidine she stated that her bladder pain was minimal. Urinary frequency was every 2.5 hours, nocturia 3 times nightly, with mild-to-moderate urinary urgency. She was advised to continue the Cetirizine-Famotidine combination, Hydroxyzine, and Gabapentin. Note that the symptomatic relief achieved by the compounded dual-histamine receptor blockade had not been achieved by prior daily treatment with Loratidine, an inhibitor of the H1 receptor.

### Example 5: A Patient with Chronic Pelvic and Bladder Pain and Treated Effectively with Dual-Histamine Receptor Blockade

A 42-year-old female who manifested at least 5 years duration of chronic pelvic and bladder pain was assessed by a physician. She reported that the symptoms began after a series of presumptive urinary tract infections, although it was uncertain whether this was diagnosed by a physician. She reported severe and painful urge to urinate, with frequent urination once per 0.5 hour (30 minutes), nocturia 2 times per night, and without incontinence. To alleviate her pain symptoms, she had been prescribed Gabapentin and Hydrocodone/Acetaminophen.

She was prescribed a compounded pharmaceutical formulation (US FDA 503A) of Cetirizine 8 mg plus Famotidine 22 mg in an orally ingested gelatin capsule that was administered once daily, in addition to Hydroxyzine 25 mg once daily. Note that Hydroxyzine, with both H1 antihistamine and antimuscarinic activities, is often prescribed for IC and/or anxiety.

At follow up by phone 3 months thereafter (by phone) she reported that her symptoms had improved by 65 percent (i.e., reduced by 65 percent), and that she was currently only administering the Cetirizine-Famotidine combination for this urologic condition. Hydroxyzine was only administered prn on occasion. She intended to continue refills of the Cetirizine-Famotidine prescription.

### Example 6: Clinical Trials Plan of Dual-Histamine Receptor Blockade in Interstitial Cystitis

To demonstrate dual-histamine receptor blockade is safe and effective in the treatment or prevention of IC, the principle clinical questions are: Can the symptoms of IC be reduced or terminated by blunting histamine's effect with dual-histamine receptor blockade? Can the treatment reduce the number of patients experiencing pain (chronic or episodic), frequent urination, and/or urinary urgency that impact upon quality of life? Can administration of the dual antihistamine therapy be effective as a preventative? Can the treatments and/or preventatives be demonstrated to be superior to standard-of-care alone (e.g., NSAIDs)?

Primary Objectives: To reduce pain, urinary frequency, or urinary urgency as symptoms of IC.

Study compounds: The H1R antagonist is cetirizine and the H2R antagonist is famotidine, wherein both active pharmaceutical ingredients (APIs) are administered orally, for gastric absorption. An alternative route of delivery for IC patients with acute symptoms might be trans-urethral canula to infuse the bladder (e.g., a separate “arm” of the study design).

Study population: Adults age 19+, male and female, with diagnosis of IC, in an outpatient environment. Females are more likely than males to participate.

Subject Groups A & B: Patients diagnosed with IC can be treated as outpatients or inpatients. The likely routes of administration are oral, however if not feasible then via trans-urethral infusion, nasogastric tube, and/or intravenous injection (IV). Group A is dual-histamine blockade and Group B is the placebo control without dual-histamine blockade. If patient disease severity dictates or an institutional review board deems that a placebo is not justifiable as the control arm, then an alternative Group B may consist of a common SOC medication (e.g., Pentosan Polysulfate or an NSAID) in lieu of a placebo control.

Sample Size: The sample size will be between approximately 20-100 patients in each arm depending on recruitment, accumulation rates, and data analysis.

If a single group of patients is treated sequentially in a cross-over design with dual-histamine blockage during one period and placebo during another period (i.e., treatment first, placebo second; or placebo first, treatment second), then a relatively low number of subjects is required. It is estimated that 20 patients receiving both test articles separately in time is sufficient to provide a 90 percent probability (p-value 0.10) at a 0.80 Standard Deviation in clinical effect. Larger groups would be required if the two groups were independent and/or the clinical effect were smaller.

Study Duration: The length of time to enroll and treat a maximum of 200 patients is estimated at 6-12 months, depending on the number of sites. If lower numbers of subjects are deemed adequate, then a shorter recruitment time. The course of patient treatment is estimated to last from approximately 2 to 8 weeks per patient, per study arm. A one-week or two-week therapy-free “wash out” period is anticipated between the two treatment periods, to minimize carryover effects.

Methodology: A randomized and blinded study design with a fixed-dose combination drug formulation is envisioned as the preferred formulation. However, alternatives could include a “two-pill” treatment with FDA-approved OTC or prescription formulations (generic or branded products) and/or prescription medications prepared by compounding pharmacy subject to 503A or 503B regulations.

Group A: Upon confirmation of diagnosis of IC, commence once daily oral Cetirizine 10 mg+Famotidine 20 mg. Alternative dosing (e.g., Cetirizine 8 mg+Famotidine 22 mg once daily) and/or alternative schedule of administration are possible.

Group B: Upon confirmation of diagnosis of IC, the control arm will receive a placebo instead of study medications (i.e., no H1 antagonist and no H2 antagonist), 1:1 age stratified relative to Group A patient population.

Exclusions: Sensitivity or allergy to H1 receptor antagonists or H2 receptor antagonists.

Primary Endpoints: (1) Reduced bladder pain, (2) reduced urinary frequency, and/or (3) reduced urinary urgency, for group A vs group B after multiple weeks of treatment.

Recording Results: The results may be recorded either electronically or on paper by the subject daily or weekly and/or at subsequent office visits with a clinician. Subjective bladder pain may be recorded using a pain scale instrument, such as a 0-10 point analog or digital scale. Urinary frequency during daytime or while awake may be recorded as the actual or subject-perceived estimated time interval between urination in hours (or minutes) during the daytime. Likewise, urinary frequency while sleeping or attempting to sleep (i.e., nocturia) may be recorded separately in a similar manner. Subjective urinary urgency may be recorded using a urinary urgency scale instrument, such as a 0-10 point analog or digital scale.

Secondary Endpoints: (1) Reduced days of routine lifestyle disruption in group A vs group B; (2) Reduced other symptoms (e.g., urinary incontinence) for group A vs group B; or (3) Reduced inflammation for group A vs group B, based upon cystoscopy or ultrasound image analysis.

Analyses: Biostatistical methods may be used to ascertain the power and statistical significance values (probabilities) for each of the primary and secondary study endpoints when comparing group A vs group B. Furthermore, each of the study endpoints may be compared post hoc to published SOC patient outcomes without the dual-histamine blockade medications, either as the control arms of the published studies or as the medication treatment arms (e.g., a single histamine receptor antagonist, and/or other pharmaceuticals, and/or biologics) of the published studies. This type of comparison may be especially informative if the number of patients is limited in the control group B, perhaps due to compassionate care or Institutional Review Board considerations limiting the number of placebo-treated patients. Thus, group A vs published SOC groups may serve as an alternative or complement to a direct group A vs group B comparison.

Anticipated Results: Using a crossover design with approximately 20 patients or more, it is anticipated that 10, 20, 40, 60, or possibly 80 percent of the subjects diagnosed with IC and treated daily with the dual antihistamine treatment will experience reduced pain relative to the same group administered the placebo. The reduction in pain, urinary frequency, and/or urinary urgency is anticipated within 1 to 2 weeks of commencing the treatment and is expected to last the duration of the course of treatment. In some patients improvements in symptom severity might manifest within 4 weeks.

The dual drug treatment might also reduce inflammation mediated by histamine, and this result can be established by cystoscopy and/or ultrasound imaging.

The dual antihistamine drug treatment might be effective in some patients only when the medication is administered continuously. In that case, ceasing treatment might result in reoccurrence of symptoms (e.g., pain). Alternatively, some patients might exhibit a long-duration clinical benefit, even after ceasing the treatment.

### REFERENCES

Akin, C., P. Valent, and D. D. Metcalfe 2010 Mast cell activation syndrome: Proposed diagnostic criteria. J Allergy Clin Immunol 126(6):1099-104 e4.
Branco, Accc, et al. 2018 Role of Histamine in Modulating the Immune Response and Inflammation. Mediators Inflamm 2018:9524075.
Chiang, G., et al. 2000 Pentosanpolysulfate inhibits mast cell histamine secretion and intracellular calcium ion levels: an alternative explanation of its beneficial effect in interstitial cystitis. J Urol 164(6):2119-25.
Dorsch, W., H. J. Reimann, and J. Neuhauser 1982 Histamine1-histamine2 antagonism: effect of combined clemastine and cimetidine pretreatment on allergen and histamine-induced reactions of the guinea pig lung in vivo and in vitro. Agents Actions 12(1-2):113-8.
el-Mansoury, M., et al. 1994 Increased urine histamine and methylhistamine in interstitial cystitis. J Urol 152(2 Pt 1):350-3.
Enerback, L., M. Fall, and F. Aldenborg 1989 Histamine and mucosal mast cells in interstitial cystitis. Agents Actions 27(1-2):113-6.
Evans, R. J. 2002 Treatment approaches for interstitial cystitis: multimodality therapy. Rev Urol 4 Suppl 1:S16-20.
Hamilton, M. J., et al. 2011 Mast cell activation syndrome: a newly recognized disorder with systemic clinical manifestations. J Allergy Clin Immunol 128(1):147-152 e2.
Hassoun, Y., M. R. Stevenson, and D. I. Bernstein 2019 Idiopathic postprandial diarrhea responsive to antihistamines. Ann Allergy Asthma Immunol 123(4):407-409.
Hogan I I, R. B., U S Patent Publication No. 2014-0275116 (2014)
Hogan I I, R. B., et al. 2020 Dual-histamine receptor blockade with cetirizine-famotidine reduces pulmonary symptoms in COVID-19 patients. Pulm Pharmacol Ther 63:101942.
Kastrup, J., et al. 1983 Histamine content and mast cell count of detrusor muscle in patients with interstitial cystitis and other types of chronic cystitis. Br J Urol 55(5):495-500.
Kmiecik, T., et al. 2012 T lymphocytes as a target of histamine action. Arch Med Sci 8(1): 154-61.
Lamale, L. M., et al. 2006 Interleukin-6, histamine, and methylhistamine as diagnostic markers for interstitial cystitis. Urology 68(4):702-6.
Lin, R. Y., et al. 2000 Improved outcomes in patients with acute allergic syndromes who are treated with combined H1 and H2 antagonists. Ann Emerg Med 36(5):462-8.
Lundeberg, T., et al. 1993 Interstitial cystitis: correlation with nerve fibres, mast cells and histamine. Br J Urol 71(4):427-9.
Lynes, W. L., et al. 1987 Mast cell involvement in interstitial cystitis. J Urol 138(4):746-52.
Mehta, Puja, et al. 2020 COVID-19: consider cytokine storm syndromes and immunosuppression. The Lancet 395(10229): 1033-1034.
Mohammadi, Ehsan, et al. 2018 287-Exploring an Antihistamine Combination Therapy for Diarrhea Predominant Irritable Bowel Syndrome. Gastroenterology 154(6):S-72.
Monroe, E. W., et al. 1981 Combined H1 and H2 antihistamine therapy in chronic urticaria. Arch Dermatol 117(7):404-7.
Pellinger, T. K., et al. 2010 Local histamine H(1-) and H(2)-receptor blockade reduces postexercise skeletal muscle interstitial glucose concentrations in humans. Appl Physiol Nutr Metab 35(5):617-26.
Phanuphak, P., A. Schocket, and P. F. Kohler 1978 Treatment of chronic idiopathic urticaria with combined H1 and H2 blockers. Clin Allergy 8(5):429-33.
Philbin, D. M., et al. 1981 The use of H1 and H2 histamine antagonists with morphine anesthesia: a double-blind study. Anesthesiology 55(3):292-6.
Runge, J. W., et al. 1992 Histamine antagonists in the treatment of acute allergic reactions. Ann Emerg Med 21(3):237-42.
Sant, G. R., et al. 2003 A pilot clinical trial of oral pentosan polysulfate and oral hydroxyzine in patients with interstitial cystitis. J Urol 170(3):810-5.
Seshadri, P., L. Emerson, and A. Morales 1994 Cimetidine in the treatment of interstitial cystitis. Urology 44(4):614-6.
Shan, H., et al. 2019 Differential expression of histamine receptors in the bladder wall tissues of patients with bladder pain syndrome/interstitial cystitis-significance in the responsiveness to antihistamine treatment and disease symptoms. BMC Urol 19(1):115.
Sielaff, T. D., et al. 1987 Successful treatment of adult respiratory distress syndrome by histamine and prostaglandin blockade in a porcine Pseudomonas model. Surgery 102(2):350-7.
Theoharides, T. C. 2007 Treatment approaches for painful bladder syndrome/interstitial cystitis. Drugs 67(2):215-35.
Valent, P. 2013 Mast cell activation syndromes: definition and classification. Allergy 68(4):417-24.
Wan, K. S. 2009 Efficacy of leukotriene receptor antagonist with an anti-H1 receptor antagonist for treatment of chronic idiopathic urticaria. J Dermatolog Treat 20(4):194-7.
Yun, S. K., et al. 1992 Stimulated release of urine histamine in interstitial cystitis. J Urol 148(4):1145-8.

1. A method of treating or preventing interstitial cystitis in a patient, comprising administering an H1 receptor antagonist and an H2 receptor antagonist to the patient.

2. A method of treating or preventing bladder pain, frequent urination, or urinary urgency in a patient affected by interstitial cystitis, comprising administering an H1 receptor antagonist and an H2 receptor antagonist to the patient.

3. A method of treating or preventing inflammation of the bladder in a patient, comprising administering an H1 receptor antagonist and an H2 receptor antagonist to the patient.

4. (canceled)

5. (canceled)

6. The method of claim 1, wherein the H1 receptor antagonist comprises cetirizine, levocetirizine, diphenhydramine, or mixtures thereof.

7. The method claim 1, wherein the H2 receptor antagonist comprises famotidine, ranitidine, cimetidine, or mixtures thereof.

8. The method claim 1, wherein the H1 receptor antagonist and the H2 receptor antagonist are administered simultaneously.

9. The method claim 1, wherein the H1 receptor antagonist and the H2 receptor antagonist are administered sequentially at intervals ranging from 2 to 12 hours.

10. The method claim 1, wherein the H1 receptor antagonist and the H2 receptor antagonist are administered by a route selected from the group consisting of injection, trans-urethral canula, by nasogastric tube, orally and mucosally.

11. The method claim 1, wherein the H1 receptor antagonist and the H2 receptor antagonist are administered once per day for at least 2 days.

12. The method, pharmaceutical composition, or use of claim 1, wherein the H1 receptor antagonist and the H2 receptor antagonist are administered once per day for at least 7 days.

13. The method claim 1, wherein the H1 receptor antagonist comprises cetirizine, and the H2 receptor antagonist comprises famotidine.

14. The method claim 1, wherein cetirizine is administered in an amount of 5 to 20 mg.

15. The method claim 1, wherein the famotidine is administered in an amount of 10 to 40 mg.

16. The method claim 1,
wherein the H1 receptor antagonist comprises cetirizine,
the H2 receptor antagonist comprises famotidine,
at most 10 mg of cetirizine is administered per day, and
at most 40 mg of famotidine is administered per day.

17. The method claim 1, wherein the patient is human.

18. The method pharmaceutical composition, or use of claim 1, wherein the H1 receptor antagonist and the H2 receptor antagonist comprise an oral dosage form.

19. The method claim 1, wherein the oral dosage form comprises a unit dosage form.

20. The method claim 1, wherein the oral dosage form comprises at least one tablet or capsule.

21. The method claim 1, wherein the oral dosage form comprises a plurality of unit dosage forms.

22. (canceled)

23. The method of claim 1, wherein the patient is a child, wherein the H1 receptor antagonist comprises cetirizine,
the H2 receptor antagonist comprises famotidine,
at most 5 mg of cetirizine is administered per day, and
at most 20 mg of famotidine is administered per day.
